(19) United States
(12) Patent Application Publication
(10) Pub. No.: US 2015/0233915 A1
(43) Pub. Date: Aug. 20, 2015

(54) BIOMARKERS FOR TUBERCULOSIS INFECTION

(71) Applicant: THE REGENTS OF THE UNIVERSITY OF MICHIGAN, Ann Arbor, MI (US)

(72) Inventor: Matthew Schaller, Ann Arbor, MI (US)

(21) Appl. No.: 14/626,046

(22) Filed: Feb. 19, 2015

Related U.S. Application Data
(60) Provisional application No. 61/942,132, filed on Feb. 20, 2014.

(51) Int. Cl. G01N 33/566; C07K 16/28; C12Q 1/68

(52) U.S. Cl. CPC G01N 33/566; C12Q 1/6806; C12Q 1/6837; C12Q 1/6872; C07K 16/28; G01N 2800/26

(57) ABSTRACT

Provided herein are compositions, kits, devices, and methods for the detection and/or diagnosis of tuberculosis (TB) infection. In particular, detection and/or quantification of notch ligand delta-like 1 (dll1) expression, notch ligand delta-like 4 (dll4) expression, or a ration thereof in a subject is used as a biomarker for diagnosis of mycobacterial infection.

### CROSS REFERENCE TO RELATED APPLICATIONS

The present application claims the benefit of U.S. Provisional Patent Application Ser. No. 61/942,132, filed Feb. 20, 2014, which is incorporated by reference in its entirety.

### FILED

Provided herein are compositions, kits, devices, and methods for the detection and/or diagnosis of tuberculosis (TB) infection. In particular, detection and/or quantification of notch ligand delta-like 1 (dll1) expression, notch ligand delta-like 4 (dll4) expression, or a ration thereof in a subject is used as a biomarker for diagnosis of mycobacterial infection.

### BACKGROUND

Tuberculosis is a clear global health problem, infecting over a third of the world population, according to the World Health Organization (WHO). The majority of these infections occur in developing countries, and to individuals without access health care facilities for proper diagnosis of TB. Current TB diagnosis relies on a number of tests fraught with problems. The tuberculosis skin tests (TST), also known as the purified protein derivative (PPD) test only tells that a person has been infected with TB bacteria. It does not tell whether the person has latent TB infection (LTBI) or has progressed toTB disease. Patients that have already been vaccinated with Bacillus Calmette-Guérin (BCG) give a false positive result when the PPD skin test is used. The Interferon Gamma Release Assay (IGRA) is both costly and unreliable for recently exposed individuals and children under the age of 5. Additionally, the IGRA requires a laboratory to run the test. Traditional diagnostic tools such as testing for acid fast bacteria in sputum and chest x-rays for the presence of granulomas can be unreliable and again require access to health facilities.

### SUMMARY

Provided herein are compositions, kits, devices, and methods for the detection and/or diagnosis of tuberculosis (TB) infection. In particular, detection and/or quantification of notch ligand delta-like 1 (dll1) expression, notch ligand delta-like 4 (dll4) expression, or a ratio thereof in a subject is used as a biomarker for diagnosis of mycobacterial infection.

In some embodiments, the present invention provides methods for diagnosing tuberculosis infection in a subject, comprising: a) detecting, in a sample obtained from the subject, the levels of expression products for notch ligand delta-like 1 (dll1) and/or notch ligand delta-like 4 (dll4); and b) diagnosing said subject as having a latent or an active TB infection based on the levels of the expression products. In some embodiments, the subject is a human subject. In some embodiments, the human subject is receiving routine screening. In some embodiments, the human subject is suspected of being at-risk for TB infection, has been exposed to TB, exhibits symptoms of TB infection, and/or is going to come in contact with a vulnerable population. In some embodiments, dll4 is the sole diagnostic determinant in said method. In some embodiments, dll4 is detected as a part of a panel of TB biomarkers. In some embodiments, the panel comprises one or more human-origin biomarkers. In some embodiments, the panel comprises one or more TB-origin biomarkers.

In some embodiments, subjects without TB infection have Dll4 levels (e.g., amount of Dll4 present, percent of circulating monocytes that are Dll4⁺, etc.) below a threshold value (e.g., <5% of total monocytes, <4.5% of total monocytes, <4% of total monocytes, <3.5% of total monocytes, <3% of total monocytes, <2.5% of total monocytes, and any ranges therein) while subjects with latent TB infection have Dll4 levels (e.g., amount of Dll4 present, percent of circulating monocytes that are Dll4⁺, etc.) above a threshold value (e.g., >3% of total monocytes, >3.5% of total monocytes, >4% of total monocytes, >4.5% of total monocytes, >5% of total monocytes, <5.5% of total monocytes, and any ranges therein). In some embodiments, subjects without TB infection have Dll4 levels (e.g., amount of Dll4 present, percent of circulating monocytes that are Dll4⁺, etc.) below a threshold value (e.g., <5% of total monocytes, <4.5% of total monocytes, <4% of total monocytes, <3.5% of total monocytes, <3% of total monocytes, <2.5% of total monocytes, and any ranges therein) while subjects with latent and/or active TB infection have Dll4 levels (e.g., amount of Dll4 present, percent of circulating monocytes that are Dll4⁺, etc.) above a threshold value (e.g., >3% of total monocytes, >3.5% of total monocytes, >4% of total monocytes, >4.5% of total monocytes, >5% of total monocytes, <5.5% of total monocytes, and any ranges therein).

In some embodiments, subjects with active TB infection have Dll1 levels (e.g., amount of Dll1 present, percent of circulating monocytes that are Dll1⁺, etc.) below a threshold value (e.g., <15% of total monocytes, <14% of total monocytes, <13% of total monocytes, <12% of total monocytes, <11% of total monocytes, <10% of total monocytes, and any ranges therein) while subjects with latent TB infection have Dll4 levels (e.g., amount of Dll4 present, percent of circulating monocytes that are Dll4⁺, etc.) above a threshold value (e.g., >15% of total monocytes, >16% of total monocytes, >18% of total monocytes, >20% of total monocytes, >22% of total monocytes, <24% of total monocytes, and any ranges therein). In some embodiments, subjects with active TB infection have Dll1 levels (e.g., amount of Dll1 present, percent of circulating monocytes that are Dll1⁺, etc.) below a threshold value (e.g., <15% of total monocytes, <14% of total monocytes, <13% of total monocytes, <12% of total monocytes, <11% of total monocytes, <10% of total monocytes, and any ranges therein) while subjects with latent or without TB infection have Dll1 levels (e.g., amount of Dll1 present, percent of circulating monocytes that are Dll1⁺, etc.) above a threshold value (e.g., >15% of total monocytes, >16% of total monocytes, >18% of total monocytes, >20% of total monocytes, >22% of total monocytes, <24% of total monocytes, and any ranges therein). In some embodiments, subjects with latent TB infection have Dll1 levels (e.g., amount of Dll1 present, percent of circulating monocytes that are Dll1⁺, etc.) above a threshold value while subjects with active TB infection or without TB infection have Dll1 levels (e.g., amount of Dll1 present, percent of circulating monocytes that are Dll1⁺, etc.) below a threshold value (e.g., the same threshold, a different diagnostic threshold).

In some embodiments, subjects with active TB infection have a Dll4:Dll1 ratio above a threshold value (e.g., >0.3, >0.35, >0.4, and ranges therein) while subjects with latent TB infection have a Dll4:Dll1 ratio below a threshold value (e.g., <0.3, <0.25). In some embodiments, subjects without active TB infection have a Dll4:Dll1 ratio below a threshold value (e.g., <0.3, <0.25) while subjects with active TB infection have a Dll4:Dll1 ratio above a threshold value (e.g., >0.3, >0.35, >0.4, and ranges therein).

In some embodiments, Dll4 level and/or percent of total monocytes expressing Dll4 distinguishes latent TB from the absence of TB infection. In some embodiments, Dll1 level and/or percent of total monocytes expressing Dll1 distinguishes latent TB from the active TB infection. In some embodiments, the ratio of Dll4 level to Dll1 level and/or ratio of the percent of total monocytes expressing Dll4 to the percent of total monocytes expressing Dll1 distinguishes active TB from the absence of TB infection. In some embodiments, the ratio of Dll4 level to Dll1 level and/or ratio of the percent of total monocytes expressing Dll4 to the percent of total monocytes expressing Dll1 distinguishes active TB from latent TB infection. In some embodiments, threshold values may be adjusted to account for the sample type, or degree of processing of the sample. For example, in some embodiments, when biomarkers (e.g., dll1, dll4, etc.) are detected in whole blood, threshold values will be adjusted (e.g., increased, decreased, etc.), to account, for example, for matrix effect.

In some embodiments, the expression products are all or a portion of dll1 and/or dll4 mRNA. In some embodiments, detecting the levels of expression products comprises exposing the sample to nucleic acid probes complementary to a portion of dll1 and/or dll4 mRNA. In some embodiments, the nucleic acid probes are covalently linked to a solid surface. In some embodiments, detecting the levels of expression products comprises the use of a detection technique selected from the group consisting of microarray analysis, reverse transcriptase PCR, quantitative reverse transcriptase PCR, hybridization analysis, and/or mass spectrometry.

In some embodiments, the expression product is dll1 and dll4 protein. In some embodiments, detecting the levels of expression products comprises exposing the sample to antibodies for the dll1 and/or dll4. In some embodiments, the antibodies are covalently linked to a solid surface.

In some embodiments, methods further comprise treatment for TB infection following diagnosis. In some embodiments, methods comprise further testing (e.g., chest x-ray) for TB infection following diagnosis with a dll1 and/or dll4 test.

In some embodiments, the present invention provides kits, reagent mixtures, or surfaces comprising reagents for detecting dll1 and/or dll4 as a biomarker for TB infection. In some embodiments, reagents are provided for detection of 20 or fewer biomarkers. In some embodiments, reagents comprise antibodies for dll1 or dll4. In some embodiments, reagents further comprise antibodies for other TB biomarkers. In some embodiments, said reagents comprise nucleic acid probes for dll1 or dll4 mRNA. In some embodiments, reagents further comprise nucleic acid probes for other TB biomarkers.

In some embodiments, the present invention provides devices comprising reagents for detection of dll1 and/or dll4, wherein upon exposure of said device to a biological sample from a subject, said device provides a diagnostic indication of whether or not the subject is infected with TB. In some embodiments, reagents comprise nucleic acid probes. In some embodiments, reagents comprise antibodies. In some embodiments, a biological sample comprises blood. In some embodiments, a blood sample is unprocessed blood. In some embodiments, less than 1.0 ml of unprocessed blood (e.g., <0.5 ml, <0.2 ml, <0.1 ml, <0.05 ml, <0.02 ml, <0.01 ml, etc.) is required and/or used to generate said diagnostic indication.

### BRIEF DESCRIPTION OF THE DRAWINGS

FIG. 1 shows expression of dll4 and dll1 in human peripheral blood correlates with mTB infection.

FIG. 2 shows expression of dll4 and dll1 in human peripheral blood correlates with mTB infection.

### DEFINITIONS

As used herein, the term “sample” is used in its broadest sense. In one sense it can refer to biological samples obtained from animals (including humans) and encompass fluids, solids, tissues, and gases. Biological samples include blood products (e.g., plasma and serum), saliva, urine, and the like. These examples are not to be construed as limiting the sample types applicable to the present invention.

The term “epitope” as used herein refers to that portion of an antigen that makes contact with a particular antibody. When a protein or fragment of a protein is used to immunize a host animal, numerous regions of the protein may induce the production of antibodies which bind specifically to a given region or three-dimensional structure on the protein; these regions or structures are referred to as “antigenic determinants”. An antigenic determinant may compete with the intact antigen (i.e., the “immunogen” used to elicit the immune response) for binding to an antibody.

The terms “specific binding” or “specifically binding” when used in reference to the interaction of an antibody and a protein or peptide means that the interaction is dependent upon the presence of a particular structure (i.e., the antigenic determinant or epitope) on the protein; in other words, the antibody is recognizing and binding to a specific protein structure rather than to proteins in general. For example, if an antibody is specific for epitope “A,” the presence of a protein containing epitope A (or free, unlabelled A) in a reaction containing labeled “A” and the antibody will reduce the amount of labeled A bound to the antibody.

As used herein, the terms “non-specific binding” and “background binding” when used in reference to the interaction of an antibody and a protein or peptide refer to an interaction that is not dependent on the presence of a particular structure (i.e., the antibody is binding to proteins in general rather that a particular structure such as an epitope).

### DETAILED DESCRIPTION

Provided herein are compositions, kits, devices, and methods for the detection and/or diagnosis of tuberculosis (TB) infection. In particular, detection and/or quantification of notch ligand delta-like 1 (dll1) expression, notch ligand delta-like 4 (dll4) expression, or a ration thereof in a subject is used as a biomarker for diagnosis of mycobacterial infection.

Experiments conducted during development of embodiments of the present invention identified dll1 and dll4 as cell surface ligands, the expression of which are altered between states of latent TB infection, active TB infection, and/or the absence of TB infection. In some embodiments, dll-4 is upregulated systemically (e.g., in bone marrow and peripheral blood) in response to (active) mycobacterial infection (e.g., in a human subject). In some embodiments, dll-1 is upregulated systemically (e.g., in bone marrow and peripheral blood) in response to (latent) mycobacterial infection (e.g., in a human subject). In certain embodiments, mycobacterial infection (e.g., active, latent) alters expression of dll1 and/or dll4 in hematopoietic cells at sites distal to infection (e.g., systemically) in humans. In some embodiments, the present invention provides low cost tests (e.g., diagnostic) that detect the presence and/or level of dll1 and/or dll4 (e.g., in peripheral blood) and diagnose TB infection in a subject (e.g., human) based thereupon. In some embodiments, a subject is characterized as having latent TB infection, active TB infection, or no TB infection based on the levels of dll1 and dll4, and the ratio thereof. Such a test is administered, for example, by a small blood draw (e.g., finger or heel stick) to collect a small amount of blood, followed by a short incubation time. In some embodiments, a test provides diagnostic results within 24 hours (e.g., <24 hrs., <16 hrs., <12 hrs., <8 hrs., <6 hrs., <4 hrs., <2 hrs., <1 hr.) of sample collection. In some embodiments, assays are provided that can be administered and diagnosis provided outside of a clinic, and/or other health care of scientific facility (e.g., a field test).

In some embodiments, dll1 and/or dll4 is detected as the sole determinant for TB infection. In some embodiments, expression levels and/or sample concentrations of dll4 above a threshold level are diagnostic for TB infection, in the absence of any other tests and/or indicators. In other embodiments, a dll4 detection/quantification assay is performed along with a second assay (e.g., IGRA, TST, etc.) in order to diagnose TB infection. In some embodiments, a dll1 detection/quantification assay is performed. In some embodiments, a positive dll1, dll4, and/or dll4:dll1 ration test indicates that further testing (e.g., chest X-ray) should be pursued. In some embodiments, dll1 and/or dll4 is detected as part of a panel of biomarkers (e.g., genes) that are individually or collectively indicative of and/or diagnostic of TB infection. In some embodiments, a biomarker panel comprises 1 (e.g., dll4), 2, 3, 4, 5, 6, 7, 8, 9, 10, 11, 12, 13, 14, 15, 16, 17, 18, 19, 20 . . . 30 . . . 40, or more biomarkers.

In some embodiments, the ratio of Dll4 to one or more other biomarkers (e.g., dll1, cytokine production, etc.) is diagnostic and/or indicative of TB infection. In some embodiments, ratio of Dll4 to one or more other biomarkers (e.g., dll1, cytokine production, etc.) discriminates between latent and active TB infection. In some embodiments, the presence of dll1 and/or dll4 on monocytes and T cell cytokine production are assessed in the peripheral blood of a subject, the ratio of dll4 expression to cytokine production, the ration of dll1 to cytokine production, and/or the ratio of dll4 to dll1 is used to assess if the patient has latent or active TB infection.

In some embodiments, dll1 and/or dll4 is detected with other biomarkers that are diagnostic of TB infection. In some embodiments, such biomarkers are genes of the infected subject (e.g., human genes), that are up- or down-regulated upon infection with TB. In certain embodiments, dll1 and/or dll4 and/or the other biomarkers of the panel distinguish between latent and active infection. In some embodiments, a panel comprises one or more TB genes that are detected within a sample from the infected subject (e.g., human). In some embodiments, such biomarkers are useful for detecting TB infection and/or determining characteristics of the particular TB infection (e.g., strain, sub-strain, virulence, antibiotic resistance, etc.). TB genes, portions of which may find use in a pane of the present invention include, but are not limited to: rpoB, katG, mabA-inhA, gyrA, gyrB, and rss.

In some embodiments the present invention provides methods for characterizing the level of gene expression of dll1 and/or dll4. In certain embodiments, the level of gene expression is detected by quantifying the amount of dll1 and/or dll4 RNA (e.g., mRNA) or dll4 protein present in a sample (e.g., a blood sample, a purified sample, etc.).

In some embodiments, the level of biomarkers is detected by quantifying the amount of biomarker mRNA in a sample (e.g., blood, plasma, serum, etc.). In some embodiments, the present invention provides methods comprising the step of exposing a sample to nucleic acid probes complementary to a portion of the mRNA of dll1 and/or dll4 and/or a panel of biomarkers indicative of and/or diagnostic of TB invention. In some embodiments the methods employ a nucleic acid detection technique (e.g., microarray analysis, reverse transcriptase PCR, quantitative reverse transcriptase PCR, and hybridization analysis).

In some embodiments, the present invention provides methods for characterizing the level of gene expression of dll1 and/or dll4 and/or a panel of biomarkers indicative of and/or diagnostic of TB invention by detecting the amount of protein (e.g., dll4 alone or with a panel of biomarkers) in a sample (e.g., in the blood). In some embodiments the present invention provides methods for quantifying the amount of one or more protein biomarkers. In some embodiments, the present invention provides methods comprising the step of exposing a sample (e.g., raw blood, processed blood, etc.) to antibodies for the biomarkers (e.g., dll1, dll4, and/or a biomarker panel comprising dll1 and/or dll4). In some embodiments, detecting above a threshold level of dll4 is diagnostic of TB infection. In some embodiments, detecting above a threshold level of dll4 and/or one or more biomarkers of a panel (e.g., comprising dll4) is diagnostic of TB. In some embodiments, a diagnostic test comprises exposing a sample (e.g., blood sample) to antibodies specific to dll1 and/or dll4 and/or a biomarker panel comprising dll1 and/or dll4 and detecting the binding of the antibodies to the biomarker(s).

In some embodiments the present invention relates to gene expression profiles (e.g., increases and/or decrease in the expression of multiple genes (e.g., including dll1 and/or dll4)) that correlate with TB infection. In some embodiments, a panel of two or more genes is analyzed (e.g., 2 genes . . . 4 genes . . . 6 genes . . . 8 genes . . . 10 genes . . . 15 genes . . . 20 genes . . . 30 genes, or more.). In some embodiments, detection and/or quantification reagents (e.g., antibodies, oligonucleotide probes, etc.) are provided that have specificity for biomarkers of TB (e.g., dll1, dll4).

In some embodiments, test samples are prepared from blood from patients (e.g., suffering from TB-like symptoms, at risk for TB, suspected of being exposed to TB, with future exposure to at-risk populations, etc.), and the test is applied to the prepared samples. It is contemplated that the differential expression levels of dll1 and/or dll4 (or a panel comprising dll1 and/or dll4) of the patient samples relative to control samples provides an expression signature diagnostic of TB infection. In some embodiments, gene expression from a test sample is compared to gene expression from a negative control sample. In some embodiments, gene expression levels from a test sample are compared to predetermined threshold levels identified (e.g., based on population averages (e.g., for patients with similar age, gender, etc.)) as “normal.” In some embodiments, an increase or decrease of biomarker level greater than 1.1-fold (e.g., 1.2-fold, 1.5-fold, 2-fold, 3-fold, 5-fold, 10-fold, 20-fold, 50-fold, 100-fold, 1000-fold, or higher) compared to “normal” levels or any increase over a normal level or threshold level is indicative of and/or diagnostic of TB infection. In some embodiments, separate indicative and diagnostic thresholds are established.

Any suitable assay may be used to assess expression and/or level of biomarker (e.g., dll4) expression products. For example, in embodiments in which biomarker mRNA is detected, assays may comprise one or more of: DNA microarrays (e.g., cDNA microarrays and oligonucleotide microarrays); transfection or cell microarrays; chemical compound microarrays; antibody microarrays; Southern and Northern blotting; mass spectrometry, sequencing, etc. Nucleic acid amplification techniques may also be used, such as: polymerase chain reaction (PCR; U.S. Pat. Nos. 4,683,195, 4,683,202, 4,800,159 and 4,965,188, each of which is herein incorporated by reference in its entirety), reverse transcription polymerase chain reaction (RT-PCR), transcription-mediated amplification (TMA; U.S. Pat. Nos. 5,480,784 and 5,399,491, each of which is herein incorporated by reference in its entirety), ligase chain reaction (LCR; Walker, G. et al., Proc. Natl. Acad. Sci. USA 89: 392-396 (1992); U.S. Pat. Nos. 5,270,184 and 5,455,166, each of which is herein incorporated by reference in its entirety), strand displacement amplification (SDA; Walker, G. et al., Proc. Natl. Acad. Sci. USA 89: 392-396 (1992); U.S. Pat. Nos. 5,270,184 and 5,455,166, each of which is herein incorporated by reference in its entirety), and nucleic acid sequence based amplification (NASBA). Those of ordinary skill in the art will recognize that certain amplification techniques (e.g., PCR) require that RNA be reversed transcribed to DNA prior to amplification (e.g., RT-PCR), whereas other amplification techniques directly amplify RNA (e.g., TMA and NASBA).

In some embodiments, biomarker (e.g., dll1 and/or dll4 or a panel comprising dll1 and/or dll4) detection comprises measuring the existence of nucleic acid encoding such biomarkers in a sample. In some embodiments, nucleic acid is detected by Northern blot analysis, or by any of a variety of hybridization assays. A variety of technologies for hybridization and detection are known and available. For example, in some embodiments, TaqMan assay (PE Biosystems, Foster City, Calif.; See e.g., U.S. Pat. Nos. 5,962,233 and 5,538,848, each of which is herein incorporated by reference) is utilized. The assay is performed during a PCR reaction. The TaqMan assay exploits the 5′-3′ exonuclease activity of the AMPLITAQ GOLD DNA polymerase. A probe comprising an oligonucleotide with a 5′-reporter dye (e.g., a fluorescent dye) and a 3′-quencher dye is included in the PCR reaction. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

In other embodiments, nucleic acid is detected using a detection assay including, but not limited to, enzyme mismatch cleavage methods (e.g., Variagenics, U.S. Pat. Nos. 6,110,684, 5,958,692, 5,851,770, herein incorporated by reference in their entireties); polymerase chain reaction; branched hybridization methods (e.g., Chiron, U.S. Pat. Nos. 5,849,481, 5,710,264, 5,124,246, and 5,624,802, herein incorporated by reference in their entireties); rolling circle replication (e.g., U.S. Pat. Nos. 6,210,884, 6,183,960 and 6,235,502, herein incorporated by reference in their entireties); NASBA (e.g., U.S. Pat. No. 5,409,818, herein incorporated by reference in its entirety); molecular beacon technology (e.g., U.S. Pat. No. 6,150,097, herein incorporated by reference in its entirety); E-sensor technology (Motorola, U.S. Pat. Nos. 6,248,229, 6,221,583, 6,013,170, and 6,063,573, herein incorporated by reference in their entireties); cycling probe technology (e.g., U.S. Pat. Nos. 5,403,711, 5,011,769, and 5,660,988, herein incorporated by reference in their entireties); Dade Behring signal amplification methods (e.g., U.S. Pat. Nos. 6,121,001, 6,110,677, 5,914,230, 5,882,867, and 5,792,614, herein incorporated by reference in their entireties); ligase chain reaction (Barnay Proc. Natl. Acad. Sci USA 88, 189-93 (1991)); FULL-VELOCITY assays; and sandwich hybridization methods (e.g., U.S. Pat. No. 5,288,609, herein incorporated by reference in its entirety). In other embodiments, the detection assay employed is the INVADER assay (Third Wave Technologies) which is described in U.S. Pat. Nos. 5,846,717, 5,985,557, 5,994,069, 6,001,567, and 6,090,543, WO 97/27214 WO 98/42873, Lyamichev et al., Nat. Biotech., 17:292 (1999), Hall et al., PNAS, USA, 97:8272 (2000), each of which is herein incorporated by reference in their entirety for all purposes).

In some embodiments, biomarker expression is detected and/or quantified by measuring protein levels (e.g., level of dll1 and/or dll4 and/or a panel comprising dll1 and/or dll4). In some embodiments, protein expression is detected by any suitable method. In some embodiments, proteins are detected by immunohistochemistry. In other embodiments, proteins are detected by their binding to an antibody raised against the protein. The generation of antibodies is described below.

In certain embodiments, antibody binding is detected by techniques known in the art (e.g., radioimmunoassay, ELISA (enzyme linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In certain embodiments, antibody binding is detected by detecting a label on the primary antibody. In another embodiment, the primary antibody is detected by detecting binding of a secondary antibody or reagent to the primary antibody. In a further embodiment, the secondary antibody is labeled. Many methods are known in the art for detecting binding in an immunoassay and are within the scope of the present invention.

In some embodiments, an automated detection assay is utilized. Methods for the automation of immunoassays include those described in U.S. Pat. Nos. 5,885,530, 4,981,785, 6,159,750, and 5,358,691, each of which is herein incorporated by reference. In some embodiments, the analysis and presentation of results is also automated. In other embodiments, the immunoassay described in U.S. Pat. Nos. 5,599,677 and 5,672,480 (each of which is herein incorporated by reference) is utilized.

The present invention provides isolated antibodies and antibody fragments against the dll1 and/or dll4 biomarkers and any other biomarker useful for the diagnosis of TB infection. Such antibodies and antibody fragments can be used, for example, in diagnostic and therapeutic methods. The antibody, or antibody fragment, can be any monoclonal or polyclonal antibody that specifically recognize dll1 and/or dll4 biomarkers and any other biomarker useful for the diagnosis of TB infection. In some embodiments, the present invention provides monoclonal antibodies, or fragments thereof. In some embodiments, the monoclonal antibodies, or fragments thereof, are chimeric or humanized antibodies. In other embodiments, the monoclonal antibodies, or fragments thereof, are human antibodies.

The antibodies of the present invention find use in experimental, diagnostic and therapeutic methods. In certain embodiments, the antibodies of the present invention are used to detect the presence or absence of dll1 and/or dll4 in a sample from a patient. In certain embodiments, the antibodies of the present invention are used to quantify dll1 and/or dll4 in a sample. In other embodiments, antibodies inhibit or enhance the function of dll1 and/or dll4 in a subject.

Polyclonal antibodies can be prepared by any known method. Polyclonal antibodies can be raised by immunizing an animal (e.g. a rabbit, rat, mouse, donkey, etc.) by multiple subcutaneous or intraperitoneal injections of the relevant antigen (a purified peptide fragment, full-length recombinant protein, fusion protein, etc.,) optionally conjugated to keyhole limpet hemocyanin (KLH), serum albumin, etc. diluted in sterile saline and combined with an adjuvant (e.g. Complete or Incomplete Freund's Adjuvant) to form a stable emulsion. The polyclonal antibody is then recovered from blood, ascites and the like, of an animal so immunized Collected blood is clotted, and the serum decanted, clarified by centrifugation, and assayed for antibody titer. The polyclonal antibodies can be purified from serum or ascites according to standard methods in the art including affinity chromatography, ion-exchange chromatography, gel electrophoresis, dialysis, etc.

Monoclonal antibodies can be prepared using hybridoma methods, such as those described by Kohler and Milstein (1975) Nature 256:495. Using the hybridoma method, a mouse, hamster, or other appropriate host animal, is immunized as described above to elicit the production by lymphocytes of antibodies that will specifically bind to an immunizing antigen. Alternatively, lymphocytes can be immunized in vitro. Following immunization, the lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen as determined by immunoprecipitation, immunoblotting, or by an in vitro binding assay such as radioimmunoassay (RIA) or enzyme-linked immunosorbent assay (ELISA) can then be propagated either in vitro culture using standard methods (Goding, Monoclonal Antibodies: Principles and Practice, Academic Press, 1986) or in vivo as ascites tumors in an animal. The monoclonal antibodies can then be purified from the culture medium or ascites fluid as described for polyclonal antibodies above.

Alternatively monoclonal antibodies can also be made using recombinant DNA methods as described in U.S. Pat. No. 4,816,567. The polynucleotides encoding a monoclonal antibody are isolated, such as from mature B-cells or hybridoma cell, such as by RT-PCR using oligonucleotide primers that specifically amplify the genes encoding the heavy and light chains of the antibody, and their sequence is determined using conventional procedures. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, monoclonal antibodies are generated by the host cells. Also, recombinant monoclonal antibodies or fragments thereof of the desired species can be isolated from phage display libraries as described (McCafferty et al., 1990, Nature, 348:552-554; Clackson et al., 1991, Nature, 352:624-628; and Marks et al., 1991, J. Mol. Biol., 222:581-597).

The polynucleotide(s) encoding a monoclonal antibody can further be modified in a number of different manners using recombinant DNA technology to generate alternative antibodies. In one embodiment, the constant domains of the light and heavy chains of, for example, a mouse monoclonal antibody can be substituted 1) for those regions of, for example, a human antibody to generate a chimeric antibody or 2) for a non-immunoglobulin polypeptide to generate a fusion antibody. In other embodiments, the constant regions are truncated or removed to generate the desired antibody fragment of a monoclonal antibody. Furthermore, site-directed or high-density mutagenesis of the variable region can be used to optimize specificity, affinity, etc. of a monoclonal antibody.

In some embodiments, of the present invention the monoclonal antibody against dll1 and/or dll4 or any other biomarkers indicative of TB infection is a humanized antibody. Humanized antibodies are antibodies that contain minimal sequences from non-human (e.g., murine) antibodies within the variable regions. Such antibodies are used therapeutically to reduce antigenicity and HAMA (human anti-mouse antibody) responses when administered to a human subject. In practice, humanized antibodies are typically human antibodies with minimum to no non-human sequences. A human antibody is an antibody produced by a human or an antibody having an amino acid sequence corresponding to an antibody produced by a human.

Humanized antibodies can be produced using various techniques known in the art. An antibody can be humanized by substituting the CDR of a human antibody with that of a non-human antibody (e.g. mouse, rat, rabbit, hamster, etc.) having the desired specificity, affinity, and capability (Jones et al., 1986, Nature, 321:522-525; Riechmann et al., 1988, Nature, 332:323-327; Verhoeyen et al., 1988, Science, 239:1534-1536). The humanized antibody can be further modified by the substitution of additional residue either in the Fv framework region and/or within the replaced non-human residues to refine and optimize antibody specificity, affinity, and/or capability.

Human antibodies can be directly prepared using various techniques known in the art. Immortalized human B lymphocytes immunized in vitro or isolated from an immunized individual that produce an antibody directed against a target antigen can be generated (See, for example, Cole et al., Monoclonal Antibodies and Cancer Therapy, Alan R. Liss, p. 77 (1985); Boemer et al., 1991, J. Immunol., 147 (1):86-95; and U.S. Pat. No. 5,750,373). Also, the human antibody can be selected from a phage library, where that phage library expresses human antibodies (Vaughan et al., 1996, Nature Biotechnology, 14:309-314; Sheets et al., 1998, PNAS, 95:6157-6162; Hoogenboom and Winter, 1991, J. Mol. Biol., 227:381; Marks et al., 1991, J. Mol. Biol., 222:581). Humanized antibodies can also be made in transgenic mice containing human immunoglobulin loci that are capable upon immunization of producing the full repertoire of human antibodies in the absence of endogenous immunoglobulin production. This approach is described in U.S. Pat. Nos. 5,545,807; 5,545,806; 5,569,825; 5,625,126; 5,633,425; and 5,661,016; herein incorporated by reference in their entireties.

This invention also encompasses bispecific antibodies. Bispecific antibodies are antibodies that are capable of specifically recognizing and binding at least two different epitopes. Bispecific antibodies can be intact antibodies or antibody fragments. Techniques for making bispecific antibodies are common in the art (Millstein et al., 1983, Nature 305:537-539; Brennan et al., 1985, Science 229:81; Suresh et al, 1986, Methods in Enzymol. 121:120; Traunecker et al., 1991, EMBO J. 10:3655-3659; Shalaby et al., 1992, J. Exp. Med. 175:217-225; Kostelny et al., 1992, J. Immunol. 148:1547-1553; Gruber et al., 1994, J. Immunol. 152:5368; and U.S. Pat. No. 5,731,168).

In certain embodiments of the invention, it may be desirable to use an antibody fragment, rather than an intact antibody, to increase tumor penetration, for example. Various techniques are known for the production of antibody fragments. Traditionally, these fragments are derived via proteolytic digestion of intact antibodies (for example Morimoto et al., 1993, Journal of Biochemical and Biophysical Methods 24:107-117 and Brennan et al., 1985, Science, 229:81). However, these fragments are now typically produced directly by recombinant host cells as described above. Thus Fab, Fv, and scFv antibody fragments can all be expressed in and secreted from E. coli or other host cells, thus allowing the production of large amounts of these fragments. Alternatively, such antibody fragments can be isolated from the antibody phage libraries discussed above. The antibody fragment can also be linear antibodies as described in U.S. Pat. No. 5,641,870, for example, and can be monospecific or bispecific. Other techniques for the production of antibody fragments will be apparent to the skilled practitioner.

It may further be desirable, especially in the case of antibody fragments, to modify an antibody in order to increase its serum half-life. This can be achieved, for example, by incorporation of a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis).

The present invention further embraces variants and equivalents which are substantially homologous to the chimeric, humanized and human antibodies, or antibody fragments thereof, set forth herein. These can contain, for example, conservative substitution mutations, for example, the substitution of one or more amino acids by similar amino acids. For example, conservative substitution refers to the substitution of an amino acid with another within the same general class such as, for example, one acidic amino acid with another acidic amino acid, one basic amino acid with another basic amino acid or one neutral amino acid by another neutral amino acid. What is intended by a conservative amino acid substitution is well known in the art.

In some embodiments, a field test or rapid diagnostic assay is provided. Such assays provide quick and/or inexpensive screening. A rapid diagnostic assay is a rapid assay meaning that the time to conduct the test from drawing of a bodily fluid to completing the test is rapid (e.g. less than 24 hours (e.g., <12 hours, <6 hours, <2 hours, <1 hour, <30 minutes, <10 minutes, <2 minutes, <1 minute)). In some embodiments, assays are provided that have diagnostic sensitivity and the specificity, but can be performed outside of a clinic or laboratory and/or without additional equipment or reagents. In some embodiments, test are performed without sample processing (e.g., on whole samples (e.g., whole blood)). In some embodiments, a test maybe performed at remote locations and in suboptimal conditions (e.g., high temperature, lack of cleanliness, by an untrained individual (e.g., self-administered), etc.) and still provide reliable diagnostic results. In some embodiments, a self-administered test is provided.

In some embodiments, an assay is contained within a single device or unit. In such embodiments, the assay is performed by exposing the device or unit to sample (e.g., blood) and observing the results (e.g., as is done with a home pregnancy test). In some embodiments, a user (e.g., the subject of the assay) draws a small amount of blood and applies it to the device, and the device provides an output indicating whether the subject is positive or negative for TB infection. In some embodiments, minimal processing steps are required to perform the test (e.g., shaking, rinsing, diluting, etc.). In some embodiments, an assay device is for home use. In some embodiments, an assay device and the reagents therein are stable and usable without refrigeration. In some embodiments, a device and the reagents therein are stable (e.g., remain usable) for greater than 6 months (e.g., >6 months, >1 year, >2 years, >5 years, etc.).

In some embodiments, assays are provided that are performed by a clinician and/or a testing facility. In some embodiments, a computer-based analysis program is used to translate the raw data generated by the detection assay (e.g., the presence, absence, or amount of dll1 and/or dll4 and/or a biomarker panel) into data of predictive value for a clinician. In some embodiments, data analysis produces a vulnerability score, responsiveness score, and/or remission score.

In some embodiments, a clinician accesses the data and/or analysis thereof using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a biopsy or a blood or serum sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a blood sample, a urine sample, etc.) and directly send it to a profiling center. Where the sample also comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (e.g., expression data), specific for the diagnostic or prognostic information desired for the subject.

In some embodiments, profile data is prepared in a format suitable for interpretation by a treating clinician and/or the test subject. For example, rather than providing raw expression data, the prepared format may represent a diagnosis, risk, or likelihood assessment for the subject. Recommendations for particular treatment options may also be provided (e.g., antibiotic therapy). The data may be displayed to the clinician by any suitable method. For example, in some embodiments, the profiling service generates a report that can be printed for the clinician (e.g., at the point of care) or displayed to the clinician on a computer monitor.

In some embodiments, a report is generated (e.g., by a clinician, by a testing center, by a computer or other automated analysis system, etc.). A report may contain test results, diagnoses, and/or treatment recommendations.

In some embodiments, a sample (e.g., blood sample) from a subject (e.g., human) is tested to determine the level of dll1 and/or dll4 biomarkers (e.g., mRNA, protein, etc.). In some embodiments, data analysis comprises comparing the level of dll1 and/or dll4 biomarkers to a threshold value or range. In some embodiments, data analysis comprises calculating a ratio of dll4:dll1 (or dll1:dll4) and comparing that ration to a threshold value or range.

In some embodiments, a sample (e.g., blood sample) from a subject (e.g., human) is tested to determine: (i) total monocyte level, (ii) level of monocytes displaying dll1, and (iii) the level of monocytes displaying dll4. In some embodiments, data analysis comprises calculating the percent of total monocytes displaying dll1. In some embodiments, data analysis comprises calculating the percent of total monocytes displaying dll4. In some embodiments, data analysis comprises calculating the ratio of the level of monocytes displaying dll1 over the level of monocytes displaying dll4 (or the ratio of the level of monocytes displaying dll4 over the level of monocytes displaying dll1). In some embodiments, data analysis comprises calculating the ratio of the percent of total monocytes displaying dll1 over the percent of total monocytes displaying dll4 (or the ratio of the percent of total monocytes displaying dll4 over the percent of total monocytes displaying dll1).

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may choose further intervention, treatment, and/or counseling based on the results. In some embodiments, the data is used for research use. For example, the data may be used to further optimize the inclusion or elimination of biomarkers as more or less useful (e.g., in a particular population (e.g., children, adolescents, adults, males, females, etc.).

Compositions for use in the methods of the present invention include, but are not limited to, probes, amplification oligonucleotides, and antibodies. Particularly preferred compositions detect the level of expression (e.g., blood mRNA level, blood protein level) of dll1 and/or dll4 and/or a panel of biomarkers. Systems and kits are provided that are useful, necessary, and/or sufficient for detecting biomarker expression. Any of these compositions, alone or in combination with other compositions of the present invention, may be provided in the form of a kit or reagent mixture. For example, labeled probes and primer pairs are provided in a kit for the amplification and detection and/or quantification of a panel of genes comprising one or more biomarkers described herein. Kits may include any and all components necessary or sufficient for assays including, but not limited to, detection reagents, amplification reagents, buffers, control reagents (e.g., tissue samples, positive and negative control sample, etc.), solid supports, labels, written and/or pictorial instructions and product information, inhibitors, labeling and/or detection reagents, package environmental controls (e.g., ice, desiccants, etc.), and the like. In other embodiments, kits comprise antibodies (e.g., primary, secondary, etc.) and detection regents (e.g., labels, buffers, etc.). In some embodiments, kits provide a sub-set of the required components, wherein it is expected that the user will supply the remaining components. In some embodiments, the kits comprise two or more separate containers wherein each container houses a subset of the components to be delivered.

In some embodiments, the present invention provides therapies for TB infection identified using the methods of the present invention. In particular, the present invention provides methods and compositions for monitoring the effects of a candidate therapy and for selecting therapies for patients. In some embodiments, self-administered tests allow a patient to track the success of their own treatment.

### EXPERIMENTAL

### Example 1

In experiments conducted during development of embodiments of the present invention, delta-like 4 expressing stem cells were isolated and transferred into congenic recipients. Based on such studies, it was demonstrated that expression of delta-like 4 is maintained over short-term hematopoiesis.

Analysis of human peripheral blood samples indicates that expression of delta-like 4 is elevated on CD14+ monocytes in the peripheral blood of individuals either actively or latently infected with Mycobacterium tuberculosis (MTB) when compared to individuals that were treated for 6 months with antibiotics. In individuals with latent infection, delta-like 4 expression positively correlated with cytokine production from T cells when peripheral blood was cultured with multiple mycobacterial antigens. This correlation was absent in actively infected individuals. These findings indicate that delta-like 4 expression is altered during mycobacterial infection in humans. Furthermore, experiments indicate that the expression of the dll4 ligand alters the T cell immune response.

### Example 2

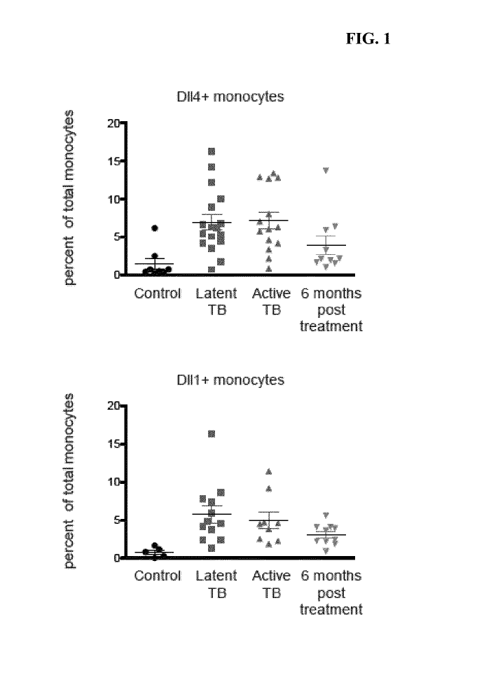

Experiments were conducted during development of embodiments of the present invention to investigate dll4 expression with mTB infection in humans. Cryopreserved peripheral blood mononuclear cells we obtained from a cohort of patients in South Africa that have been diagnosed with either latent or active TB infection by standard means. Flow cytometric analysis of these samples was performed and it was found that monocytes express more dll4 in TB infected patients than in normal healthy donors. The expression of the marker is decreased 6 months after effective treatment for TB. Increased expression of dll1, another Notch ligand that is known to be increased on myeloid cells during infection, was also observed. Data (See FIG. 1) indicates that this ligand is also upregulated on monocytes during mTB infection.

The data indicates that assessing the expression of the ligand dll4 on the cell surface, either alone or in combination with other Notch ligands, in patients infected with mTB is a useful diagnostic tool for presence of mTB infection. As the expression of dll4 resolves with infection, such a test would be used instead of the PPD skin test, which cannot be used in people that have been vaccinated with BCG. In addition, as the test requires only the presence or absence of a cell surface marker, it does not require cells to be cultured, as the IGRA does. This has significant advantages including eliminating the need for laboratory facilities, quicker time to diagnosis, and reduced cost.

### Example 3

Experiments were conducted during development of embodiments described herein to compare Dll1⁺ and Dll4⁺ monocyte levels in subjects with latent TB infection, active TB infection, and a non-TB control group (e.g., subjects having received six month of antibiotic TB treatment). 3.0×10⁶ peripheral blood mononuclear cells were incubated with human IgG and antibodies to CD14, DLL1 and DLL4 for 20 minutes at room temperature in 200 uL PBS+1% HSA on an orbital shaker at 40 RPM in a 96 well plate. After incubation, cells were washed 2× in 300 uL of PBS+1% HSA. Cells were then fixed with 100 uL normal buffered formalin for 10 minutes and run on an LSRII flow cytometer with 488, 633 and 405 nM lasers. Approximately 200,000 events were collected per sample. Samples were analyzed using FlowJo X. Samples were first gated using FSC and SSC to enrich for lymphocytes. A CD14×SSC gate was used to gate on CD14+ cells, and then cells gated on Dll1+ or Dll4+ cells. To stain for CD14, monoclonal antibody ME52 was used. For DLL4, monoclonal antibody MHD4-46 (Biolegend) was used, and for DLL1, clone 251127 (R&D systems) was used.

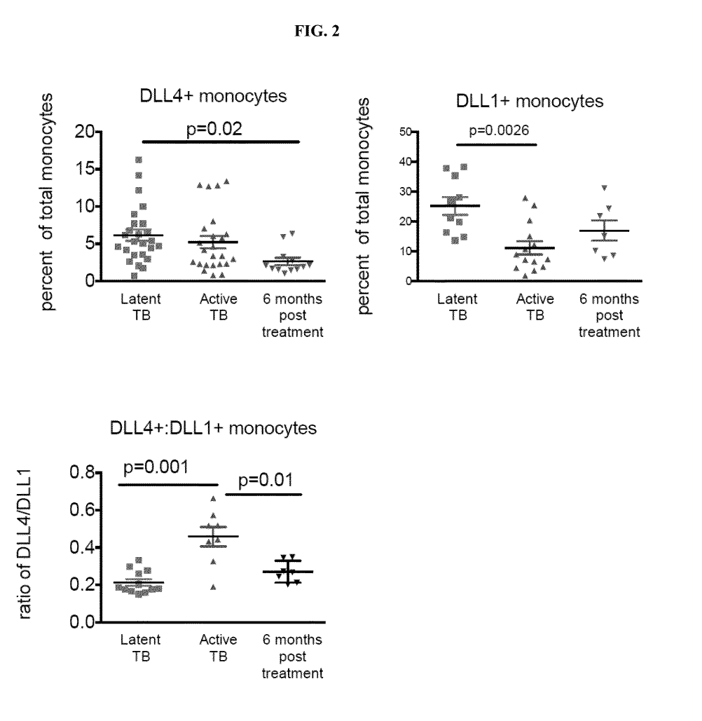

One-way ANOVA analysis was conducted on a cohort of cryopreserved peripheral blood mononuclear cells (PBMCs) from patients diagnosed with latent or active TB (FIG. 2). A group of patients treated for 6 months with appropriate antibiotics was also analyzed. A significant difference in the level of Dll4+ monocytes distinguished latent TB from treated individuals. A significant difference the level of Dll1+ monocytes distinguished latent TB from active TB. The ratio of Dll4+:Dll1+ monocytes distinguished active TB from both latent and treated patients.

1. A method for diagnosing tuberculosis (TB) infection in a subject, comprising:
(a) detecting, in a sample obtained from the subject, the levels of expression products for notch ligand delta-like 1 (dll1) and notch ligand delta-like 4 (dll4); and
b) diagnosing said subject as having a latent TB infection or an active TB infection based on the levels of the expression products.

2. The method of claim 1, wherein the subject is a human subject.

3. The method of claim 2, wherein the human subject is receiving routine screening.

4. The method of claim 2, wherein the human subject is suspected of being at-risk for TB infection, has been exposed to TB, exhibits symptoms of TB infection, and/or is going to come in contact with a vulnerable population.

5. The method of claim 1, wherein dll1, dll4, or both dll1 and dll4 is the sole diagnostic determinant in said method.

6. The method of claim 1, wherein all1 and/or dll4 is detected as a part of a panel of TB biomarkers.

7. The method of claim 6, wherein said panel comprises one or more human-origin biomarkers.

8. The method of claim 6, wherein said panel comprises one or more TB-origin biomarkers.

9. The method of claim 1, wherein the expression products are all or a portion of dll1 and/or dll4 mRNA.

10. The method of claim 9, wherein detecting the levels of expression products comprises exposing the sample to nucleic acid probes complementary to all or a portion of dll1 and/or dll4 mRNA.

11. The method of claim 10, wherein the nucleic acid probes are covalently linked to a solid surface.

12. The method of claim 9, wherein detecting the levels of expression products comprises the use of a detection technique selected from the group consisting of microarray analysis, reverse transcriptase PCR, quantitative reverse transcriptase PCR, hybridization analysis, and/or mass spectrometry.

13. The method of claim 1, wherein the expression product is dll1 and/or dll4 protein.

14. The method of claim 13, wherein detecting the levels of expression products comprises exposing the sample to antibodies, aptamers, or antibody fragments for dll1 and/or dll4 protein.

15. The method of claim 14, wherein the antibodies are covalently linked to a solid surface.

16. The method of claim 1, further comprising (c) administering therapy to treat said TB infection.

17. A kit, reagent mixture, or surface comprising reagents for detecting dll4 as a biomarker for TB infection.

18. The kit, reagent mixture, or surface of claim 17, wherein said reagents comprise antibodies for dll1 and/or dll4.

19. The kit, reagent mixture, or surface of claim 17, wherein said reagents comprise nucleic acid probes for dll1 and/or dll4 mRNA.
